SEQUENCE LISTING (7 sequences)

SEQ ID NO: 1 - DNA, 25 bp, Escherichia coli
cgttatacggaacgctccagcgttt

SEQ ID NO: 2 - DNA, 25 bp, Escherichia coli
tagggaaagaacaatggcggttgcg

SEQ ID NO: 3 - DNA, 25 bp, Escherichia coli
gaagccaaacgccagcgctcacttc

SEQ ID NO: 4 - DNA, 25 bp, Escherichia coli
caaaaacgtcgtcttttcggcggct

SEQ ID NO: 5 - DNA, 26 bp, Escherichia coli
aagtggcttcaagtacggtcaggtcg

SEQ ID NO: 6 - DNA, 25 bp, Escherichia coli
aaacgctggagcgttccgtataacg

SEQ ID NO: 7 - DNA, 25 bp, Escherichia coli
gcaatatgccttgcgaggtcgcaaa

(19) United States
(12) Patent Application Publication
(10) Pub. No.: US 2006/0228738 A1
(43) Pub. Date: Oct. 12, 2006

(54) DNA-polypyrrole based biosensors for rapid detection of microorganisms

(75) Inventors: Evangelyn Alocilja, East Lansing, MI (US); Maria I. Rodriguez-Lopez, Dearborn Heights, MI (US)

Correspondence Address: Ian C. McLeod;McLeod & Moyne, P.C., 2190 Commons Parkway, Okemos, MI, 48864, US

(73) Assignee: Board of Trustees of Michigan State University, East Lansing, MI (US)

(21) Appl. No.: 11/402,034

(22) Filed: Apr. 11, 2006

Related U.S. Application Data
(60) Provisional application No. 60/670,110, filed on Apr. 11, 2005.

(51) Int. Cl. C12Q 1/68; C12M 1/34

(52) U.S. Cl. 435/6; 435/287.2; 205/777

(57) ABSTRACT

A DNA-polypyrrole based biosensor and methods of using the biosensor for the rapid detection of Escherichia Coli and other microorganisms. The DNA-polypyrrole biosensor can be used to detect dangerous micoorganisms for monitoring water quality of a sample from a drinking water or food source. The biosensor can use genomic DNA extracted from natural environments in field settings for the rapid detection of microorganisms to provide an early warning of water contamination.

### CROSS-REFERENCE TO RELATED APPLICATIONS

This application claims priority to U.S. Provisional Patent Application No. 60/670,110, filed Apr. 11, 2005.

### STATEMENT REGARDING FEDERALLY SPONSORED RESEARCH OR DEVELOPMENT

Not Applicable

Reference to a “Computer Listing Appendix Submitted on a Compact Disc”

The application contains nucleotide sequences which are identified with SEQ ID NOs. A compact disc is provided which contains the Sequence Listings for the sequences. The Sequence Listing on the compact disc is identical to the paper copy of the Sequence Listing provided with the application.

### BACKGROUND OF THE INVENTION

(1) Field of the Invention

The present invention relates generally to biosensors, and more particularly to DNA-polypyrrole based biosensors for the detection of DNA in water samples. Specifically, the present invention relates to DNA-polypyrrole based biosensors for rapid detection of Escherichia coli and other microorganisms in the water samples from drinking water or foods, particularly at very low contamination levels.

(2) Description of the Related Art

The U.S. water supply has been identified as a potential target for bioterrorist attacks. With no routine monitoring for suspect agents and no federal treatment protocols in place, water utilities are not prepared for possible acts of bioterrorism. The spread of anthrax in 2001 has proven that any bioterrorist attacks is a potential reality at any time and there is a need be prepared for it. The use of rapid detection systems, such as biosensors, could prevent such occurrence from causing harm to humans in a disastrous scale. Food safety threats, such as Salmonella, Escherichia coli, Shigella, botulism (Clostridium botulinum), and anthrax (Bacillus antracis) are some of the bioterrorist pathogens that the Centers for Disease Control and Prevention (CDC) has identified as potential threats to the water distribution systems as well. (http://www.bt.cdc.gov/agent/agentlist.asp. Centers for Disease Control and Prevention. Emergency Preparedness and Response). These organisms can get to the water supply easily by direct contamination or by contaminated cattle waste. Some other water threats, such as Vibrio cholerae, Crystosporidium parvum and E. coli species are also included in the CDC's list of category A and B biodefense agents. Besides its national security application, the same biosensor system might be developed for the purpose of assessing water quality on an everyday basis in the future. Unprecedented interest in the development of analytical devices for rapid detection and monitoring of chemical and biological species has led to the emergence of these biosensors. The biosensor technology promises to offer new detection alternatives for pathogenic bacteria. For example, an integrated optic interferometer for detecting Salmonella typhimurium with sensitivity of 10⁵-107 colony forming units per milliliter (cfu/ml) has been developed (Seo, K. H. et al., Journal of Food Protection 62(5): 431-437 (1999)). A luminescence-based method could detect 10²-10³ cfu of E. coli 0157:H7 and Salmonella typhimurium in fresh produce (Mathew, F., et al., Proceedings of the IEEE Sensors Conference, Orlando, Fla. 12-14 (2002)).

Conductive polymers, such as polypyrrole (PPY) and polyaniline (PANI), have been extensively researched for their application in biosensors. These types of materials exhibit interesting and promising electrical and optical properties only exhibited in inorganic materials. Both have a relatively high conductivity and good environmental stability (Kanga, E. T., et al., Progress in Polymer Science 23(2) 277-324 (1998)). The principal reason for the conductive nature of some polymers are the presence of a negative (−) electron backbone with single and double bonds alternating along the polymer chain. The double strands of DNA also exhibit this electron backbone configuration, facilitating faster electron transfer along the DNA chains (Kelley, S. O., et al., Sciences 283(5400) 375-381 (1999)) and therefore to the conductive polymer. Polypyrrole is a polyheterocycline that has been extensively studied as a conductive polymer-forming film (Kanazawa, K. K., et al., J. Chem. Soc. Chem. Commun., 854-855 (1979). Some of its applications include the electrochemical deposition onto n-silicon for solar cell fabrication (Audebert, P., et al., J. Electroanal. Chem. 190 129-139 (1985). Some studies have used PPY films in a neurotransmitter as a drug release into the brain (Zinger, B., et al., J. Am. Chem. Soc. 106 6861-6863 (1984).

Current detection methods for water quality monitoring are very sensitive but require twenty-four (24) to forty-eight (48) hours for confirmation and are labor intensive (APHA 1998. Standard methods for the Examination of Water and Wastewater. 20ed. Washington, D.C.). Routine and widely accepted classical detection techniques including multiple-tube fermentation (MTF) and membrane filtration (MF) techniques. Both use specific media and the incubation period is twenty-four (24) to forty-eight (48) hours (AFNOR. 1990. Eaux-methods d'essais. Recueil de Normes Franzaided 4^{th} ed Paris: Ia Defense). Other classical detection techniques include enzymatic methods, such as the detection of β-D galactosidase and β-D glucuronidase (Kilian, M. P., et al., Acta Pathol. Microbiol. Scand., Sect. B 84:245-251 (1976) and Hartman, P. A., Rapids Methods and Automatation in Microbiology and Immunology 209-308 (1989)). Enzymatic techniques are expensive and, even though their incubation period is shorter than the classical culture techniques, they still are not fast enough for same-day results. Several molecular techniques have been developed, such as the polymerase chain reaction (PCR) and DNA-DNA hybridization which require expensive reactants, fluorescent probes or radioactivity.

Korri-Youssoufi et al. (Korri-Youssoufi, H., et al., J. Am. Chem. Soc. 119 7388-7389 (1197)) teach an oligonucleotide-functionalized polypyrrole electrode which has a voltammetric response when incubated for two hours with a complementary oligonucleotide under certain conditions, while having no response when incubated with a noncomplementary oligonucleotide. Korri-Youssoufi et al. does not teach an apparatus or methods for detecting the presence of Escherichia coli and other microorganisms of interest in drinking water or food sources.

Wang, J., et al., Analytical Acta 402: 7-12 (1999) teach doping of electrodes with oligo(dA)₂₀, oligo(dC)₂₀, oligo(dG)₂₀, or oligo(dT)₂₀ oligonucleotide probes within electropolymerized polypyrrole (PPY) films. Wang, J., et al. show that the electrodes exhibit transient current responses with addition of complementary or non-complementary oligonucleotides. Addition of complementary oligonucleotides and non-complementary oligonucleotides to the electrodes result in transient current responses with opposite directions.

While the related art teach the application of conductive polymers, such as polypyrrole (PPY) and polyaniline (PANI), in biosensors, there still exists a need for DNA-polypyrrole based biosensors capable of rapidly detecting Escherichia coli and other microorganisms in water samples.

### OBJECTS

Therefore, it is an object of the present invention to provide a DNA-polypyrrole based biosensor.

It is further an object of the present invention to provide a biosensor which can rapidly detect Escherichia coli or other microorganisms in water samples.

These and other objects will become increasingly apparent by reference to the following description.

### SUMMARY OF THE INVENTION

The present invention provides a biosensor electrode for the detection of the presence in a water sample from a drinking water or food source of a nucleic acid from a bacteria of interest comprising: an electrode; polypyrrole electropolymerized on the electrode; and an oligonucleotide as a dopant bonded to the polypyrrole having an oligonucleotide nucleotide sequence, wherein at least a portion of the oligonucleotide sequence is complementary to a unique target sequence of the nucleic acid from the bacteria of interest such that the nucleic acid from the bacteria hybridizes to the oligonucleotide for detecting the presence of the bacteria of interest by a change in conductivity of the electrode. In further embodiments the target nucleic acid is DNA or RNA. In still further embodiments the oligonucleotide comprises a DNA sequence from the uidA gene of E. coli. In still further embodiments the electrode is platinum.

The present invention provides a biosensor system for the rapid detection of the presence in a water sample from a drinking water or food source of a nucleic acid from a bacteria of interest comprising: an electrode; polypyrrole electropolymerized on the electrode; an oligonucleotide as a dopant bonded to the polypyrrole having an oligonucleotide nucleotide sequence, wherein at least a portion of the oligonucleotide sequence is complementary to a unique target sequence of the nucleic acid from the bacteria of interest such that the nucleic acid from the bacteria hybridizes to the oligonucleotide for detecting the presence of the bacteria of interest by a change in conductivity of the electrode; and an electrical detection apparatus, wherein when the oligonucleotide hybridizes to the nucleic acid an electrical signal as a result of a change of conductivity of the electrode is generated which is used to detect the presence of the bacteria of interest in the water sample. In further embodiments the target nucleic acid is DNA or RNA. In still further embodiments the oligonucleotide comprises a DNA sequence from the uidA gene of E. coli. In still further embodiments the electrode is platinum. In further embodiments the detection apparatus is a potentiostat.

The present invention provides a method of using a biosensor system for rapidly detecting the presence in a water sample from a drinking water or food source of a nucleic acid from a bacteria of interest comprising: providing a biosensor system comprising an electrode, a polypyrrole electropolymerized on the electrode, an oligonucleotide as a dopant bonded to the polypyrrole having an oligonucleotide nucleotide sequence, wherein at least a portion of the oligonucleotide sequence is complementary to a unique target nucleic acid sequence of the nucleic acid from the bacteria of interest such that the nucleic acid hybridizes to the oligonucleotide when detecting the presence of the bacteria of interest, and a detection apparatus; providing a water sample to be tested; adding a lysis solution to the water sample so as to rupture any bacteria present in the water sample to provide a prepared water sample; providing the prepared water sample to the electrode of the biosensor system; and generating a signal with the detection apparatus; and analyzing the delta charge value (ΔQ) of the signal so as to detect whether the nucleic acid from the bacteria of interest is present in the water sample.

In further embodiments the target nucleic acid is DNA or RNA. In still further embodiments the oligonucleotide comprises a DNA sequence from the uidA gene of E. coli. In still further embodiments the electrode is platinum. In further embodiments the detection apparatus is a potentiostat generating a cyclic voltammogram.

### BRIEF DESCRIPTION OF THE DRAWINGS

FIG. 1 is a schematic view which illustrates one embodiment of the present invention showing a configuration of an electrochemical cell 10 having a biosensor electrode 11.

FIG. 2 is a schematic view which illustrates a proposed model of the modified DNA-based biosensor. Upon the platinum electrode (Pt electrode) is a polypyrrole (PPY) layer bound to the oligonucleotide probe (uidA probe) which is capable of hybridizing to complementary DNA (C-uidA) from a bacteria of interest.

FIG. 3 is a graph showing polymerization of 0.05M PPY/0.5M KCl onto Pt electrode. Cyclic voltammograms after (a) 26 cycles; and (b) 13 cycles between 0.0 and 0.7 V at a scanning rate of 50 mV/s.

FIG. 4 is a graph showing a comparative cyclic voltammogram (CV) electro-deposition for 1 μg of total DNA. Cyclic voltammograms after 26 cycles between 0.0 and 0.7 V at a scanning rate of 50 mV/s for: (a) a blank solution 0.1M glycine/0.1M NaCl; (b) polymerization of PPY 0.05M/0.5M KCl; (c) complementary uidA; and (d) non-complementary uidA probe (1 μg total) in 0.1M glycine/0.1M NaCl.

FIG. 5 is a graph showing subtractive CV of complementary and non-complementary oligonucleotides targeting E. coli uidA gene fragment. Potential range from 0.0 and 0.7 V, scanning rate of 50 mV/s in 0.05 M PPY/0.5M KCl cyclic voltammograms after 26 cycles.

FIG. 6 shows scanning electron microscopy (SEM) of: (A) bare platinum (Pt); and (B) modified electrode surface.

FIG. 7 shows a two-dimensional surface profile of the PPY at different contrast (A and B).

FIG. 8 is a graph illustrating subtractive CVs for different concentrations of Complementary and Non-Complementary Oligonucleotides. Hybridization temperature was 72° C.

FIG. 9 is a graph illustrating subtractive CV of all complementary and non-complementary oligonucleotides at different concentrations.

FIG. 10 is a graph illustrating CVs for 1 μg of total complementary oligonucleotides at different hybridization times.

FIG. 11 is a graph illustrating CVs for 100 ng of complementary oligonucleotides at different hybridization times.

FIG. 12 is a histogram showing the average ΔQ for 1 μg of complementary and non-complementary probes at different hybridization times.

FIG. 13 is a histogram showing the average ΔQ of 100 ng of complementary and non-complementary probes at different hybridization times

FIG. 14 is a histogram showing a comparison of average μQ (delta charge in mC) values with respect to oligonucleotide concentrations and hybridization times: T30=thirty minutes, T60=sixty minutes, T180=one hundred eighty minutes.

FIG. 15 is an AFM image of a Pt-PPY film prior to the embedding of uidA oligonucleotides.

FIG. 16 is an AFM image of the functionalized Pt-PPY-uidA biosensor after embedding of the 25 bp oligonucleotides.

FIG. 17 is an AFM image of Pt-PPY-uidA biosensor after hybridization with complementary uidA probe.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

As used herein, the term “PPY” refers to polypyrrole.

As used herein, the term “CV” refers to a cyclic voltammogram. Cyclic voltammograms (CVs) of current (I) vs. potential (V/Ag/AgCl) are used to evaluate the performance of the biosensor.

As used herein, the term “rapid” or “rapidly” refers to times of thirty minutes or less.

Electropolymerization is an effective technique for the deposition of polymer coatings onto various substrates (Su, W., et al., Electrochimica Acta 44(13): 2173-2184 (1999) such as Pt. Au, Fe, Al, stainless steel and carbon fibers. Electrical conductivity is a measure of the capacity of a material to conduct electricity. The electrical conductivity of PPY has been demonstrated to be in the range of 10⁻³ to 10³ S cm⁻¹ (Diaz A F, Bargon. J. Electrochemical synthesis of conducting polymers. In: Skoteim T A, editor. Handbook of conducting polymers. New York: Marcel Dekker, 1986, 1:81). Electrical conduction in PPY is the result of electron movement within delocalized orbitals and positive charge defects known as polarons (Devreux, F., et al., B. Synth. Met. 18: 89 (1987). These positive charges are located every three or four pyrrole monomers along the polymer backbone and is the place were negatively charged dopants (DNA in this case) will be deposited (Satoh M., et al Met. 14 289 (1986)). Therefore DNA can form a bond with PPY based on the interchanging of dopant molecules within PPY and negatively charged biomolecules such as DNA (Boyle, A., et al., Chem. 279 179 (1990)). Hydrogen bonding to phosphate oxygen in the DNA backbone can enhance binding to DNA. PPY will provide the hydrogen bonds through its nitrogen atoms. The use of free standing PPY films exposed to radioactive label ³²p double strand DNA demonstrated the adsorption kinetics of DNA-PPY films (Mineham, D. S., et al., Macromolecules 27 777-783 (1994) and Pande, R., et al., Biomaterials 19 1657-1667 (1998)). These studies demonstrated that DNA uptake exhibited a t^{1/2} dependence. These results were for adsorption of DNA into a PPY without the use of cyclic voltammetry. The methods of the present invention take advantage of the DNA adsorption onto the polymer film in a real time manner due to the voltage application during the cyclic voltammetric electro-deposition.

The present invention uses polypyrrole in a DNA based E. coli model biosensor for fast and accurate water quality monitoring in field settings, particularly for early warning of water contamination. Prior to this, no one has proven the effectiveness of a Pt-PPY-DNA system using environmental DNA samples. The specificity and stability of the Pt-DNA-PPY biosensor is shown herein. Unlike other DNA-conductive polymer designs (Korri-Youssoufi, H., et al., J. Am. Chem. Soc. 119 7388-7389 (1197)), the E. coli DNA biosensor of the present invention uses genomic DNA which has been extracted from natural environments as a target for analysis instead of a synthetic DNA oligonucleotide. Five different twenty-five (25) base pair (bp) oligonucleotide probes have been used for testing of an embodiment of a biosensor system utilizing the uidA gene from E. coli strain K-12 E. coli for the detection of hazardous biological agents in water contamination. Unlike other DNA-conductive polymer designs, the E. coli DNA biosensor can use genomic DNA extracted from natural environments as target instead of a synthetic DNA oligonucleotide. Studies have proven that a 126 bp complementary fragment from the uidA gene of E. coli is capable of hybridizing with fifty different strains of E. coli present in fresh water (Farnleitner, A. H., et al., Appl. Envir. Microbiol. 1340-1346 (2000)).

Some advantages of the present invention are: that it is label free (i.e. no need for expensive fluorescent dyes or radioisotopes); it gives same day results; it has molecular specificity; it can be adapted to different bio-terrorist agents; it is inexpensive and does not require expensive lab equipment. Homeland Security Agencies, Drinking Water Processing Facilities, Food Processing Facilities, and Clinical Diagnostics Laboratories can all utilize the present invention.

The principal purpose of this invention is to provide a highly specific, sensitive, real-time DNA-based biosensor for the potential detection of Escherichia coli as a representative of fecal coliforms in water. The biosensor is faster and more cost efficient than most of the current detection methods. Molecular biology and chemical electrode position techniques such as cyclic voltammetry were combined to develop and test the DNA-based biosensor. In one embodiment of the present invention a Platinum (Pt) electrode is electro-polymerized with polypyrrole (PPY), a conductive polymer and the complementary oligonucleotide. The recognition element is a twenty-five (25) base pair (bp) oligonucleotide specific for E. coli derived from the uidA gene that codes for the enzyme β-D-glucuronidase. Nucleic acids isolated from pure E. coli cultures and from water samples serve as the analyte. In this embodiment, the device incorporates the complementary uidA gene oligonucleotide into a conductive: polymer-electrode biosensor system, particularly a polypyrrole-coated platinum electrode. The sensitivity of the DNA biosensor can be determined by using different concentrations of DNA extracted from E. coli pure culture. The specificity of the biosensor can be determined using DNA from common waterborne pathogenic microorganisms. The specificity testing can be performed using genomic DNA from E. coli pure culture as well as from other related Enterobacteria genomic DNA. Analysis using total DNA extracted from water samples can also be performed. The stability of the biosensor is determined in the presence of common organic water pollutants and organic matter.

The biosensor is capable of generating distinctive cyclic voltammograms (CV) for complementary and non-complementary DNA sequences. In the Examples, cyclic voltammetric scannings between 0.0 and +0.70V and with a 50 m V/s scanning rate are used to generate current vs. potential graphs. Standard total DNA concentrations ranging from 1 microgram (μg) to 1 nanogram (ng) were used to determine a hybridization signal. Lower DNA concentrations closer to environmental conditions were used to determine the sensitivity limit of the biosensor. Genomic DNA from other enteric pathogenic species and from natural and recreational water samples can be used. The present invention is useful as a fast, sensitive and specific DNA based biosensor which is of great utility for environmental monitoring and policy formulation.

The following Examples show that the biosensor is an efficient and capable biosensor. The hybridization capability of embedded DNA into polypyrrole (PPY) with complementary DNA samples was determined. In the biosensor platform described in the following Examples the Platinum (Pt) electrode electropolymerized with PPY. Electrodes constructed of other conductive materials, such as other metals, are also encompassed by the present invention. The recognition elements of a preferred embodiment were oligonucleotides specific for Escherichia coli derived from the uidA gene that codes for the enzyme for the β-D-glucuronidase. The biosensor was capable of generating distinctive cyclic voltammetric signals for complementary and non-complementary DNA sequences. Cyclic voltamometric scanning between 0.0 and +0.70 V with a 50 mV/s scanning rate were used to generate current vs. potential graphs. A standard DNA concentration of 1 μg/μl was used to determine the signal hybridization signal recognition of the biosensor. The biosensor platform proved to be effective in the detection of complementary uidA 25 bp oligonucleotide and genomic DNA for E. coli K-12. The Examples demonstrated the potential for using the fast, sensitive and specific DNA based biosensors of the present invention for the detection of pathogenic bacteria in natural and drinking water sources.

### EXAMPLES

Selection of the DNA sequence for the detection of E. coli. The gene that codes for the enzyme β-D-glucuronidase known as uidA was selected for identification of diverse aquatic strains of E. coli. The sequence for the uidA gene was obtained from the public data base GenBank (accession no. M14641). Studies have proven the use of a 126 bp fragment from the 1640 to 1805 position for its ability to hybridize with 50 different strains of E. coli present in fresh water (Farnleitner, A. H., et al., Appl. Envir. Microbiol. 2000: 1340-1346 (2000)). Five twenty-five base pair (25 bp) oligonucleotides from E. coli K-12 uidA gene positions 1640 to 1805 (Table 1) were synthesized. The synthesis of the oligonucleotides were carried out at the Genomics Technology Support Facility at Michigan State University (http://genomics.msu.edu, Michigan State University Genomics Technology Support Facilities). A 25 bp synthetic oligonucleotide with the sequence 5′-CGTTATACGGAACGCTCCAGCGTTT-3′ (SEQ ID NO:1) is used as the embedded probe. Two other oligonucleotides were synthesized to be used as complementary target (5′-AAACGCTGGAGCGTTCCGTATAACG-3′) (SEQ ID NO:6) and as non-complementary target (5′-GCAATATGCCTTGCGAGGTCGCAAA-3′)(SEQ ID NO:7).

TABLE 1

Sequence of the five 25-26 bp probes for the
detection of E. coli from water samples.

| | | | |
|---|---|---|---|
| | | | SEQ |
| | LENGTH | | ID |
| PROBE | (BP) | SEQUENCE | NO |

| | | | |
|---|---|---|---|
| 1 | 25 | 5′-cgttatacggaacgctccagcgttt-3′ | 1 |
| 2 | 25 | 5′-tagggaaagaacaatggcggttgcg-3′ | 2 |
| 3 | 25 | 5′-gaagccaaacgccagcgctcacttc-3′ | 3 |
| 4 | 25 | 5′-caaaaacgtcgtcttttcggcggct-3′ | 4 |
| 5 | 26 | 5′-aagtggcttcaagtacggtcaggtcg-3′ | 5 |

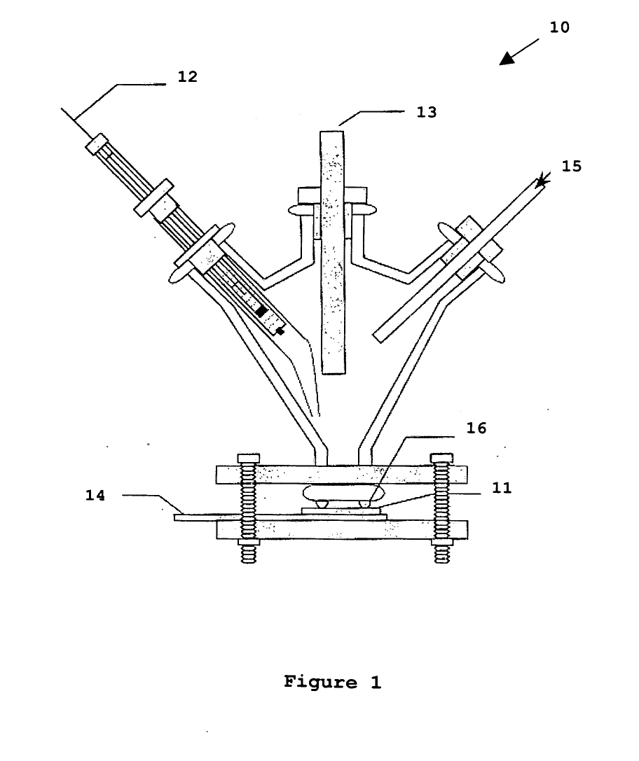

The preparation of the Pt-PPY-oligonucleotide E. coli biosensor. The biosensor design is a modification of the DNA-based oligonucleotide-functionalized PPY used by Korri-Youssouffi et al. The three electrode cell 10 used is illustrated in FIG. 1, courtesy of Dr. Greg Swain's Laboratory, Department of Chemistry, Michigan State University. The three electrode cell 10, comprising of a platinum working electrode 11 having a 3 mm diameter, a Ag/AgCl (3 M NaCl) reference electrode 12, and a carbon rod counter electrode 13, was placed against a copper foil plate 14 and connected to a potentiostat/galvanostat (Versastat Model II, Princeton Applied Research, Oak Ridge, Tenn.). The electrochemical response can be measured using the potentiostat/galvanostat to generate cyclic voltammograms. The electrochemical cell 10 has a N₂/O₂ gas inlet 15, and has a Viton o-ring 16 as a seal against the platinum working electrode 11. The electrochemical cell 10 has a total volume of two milliliters (2 ml) having 0.05 M distilled pyrrole (Sigma-Aldrich, St. Louis Mo.) and 2 μl of a 500 μg/ml solution (for a total of 1 μg) of oligonucleotide probe. The electro-polymerization was achieved by a continuous cyclic voltammetry scanning between 0.0 and +0.70V at a scan rate of 50 mV/s. The potentiostat performed for 10 cycles. Following electro-polymerization, the modified surface was rinsed with sterilized water. Measurement of background signal was performed by cyclic voltammetry with a blank electrolyte solution (1M KCl).

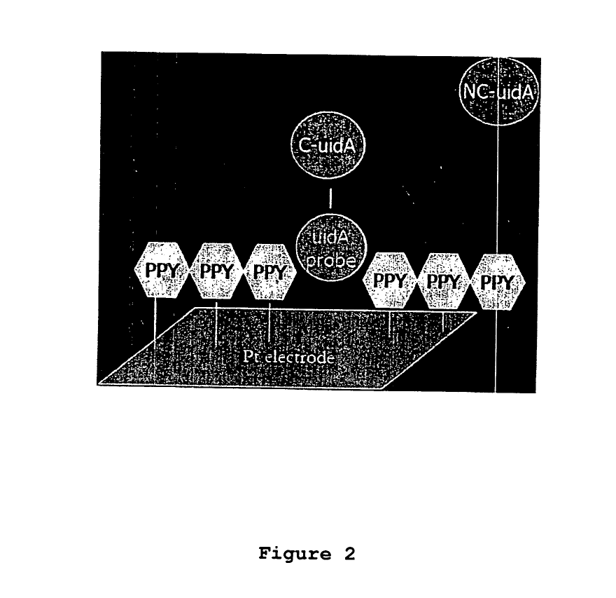

A schematic of how the E. coli biosensor functions is illustrated in FIG. 2. Upon the platinum working electrode (Pt electrode) of the E. coli biosensor is a polypyrrole (PPY) layer that is bound to an oligonucleotide probe that is specific for an E. coli gene such as the uidA gene (uidA probe). The oligonucleotide probe (uidA probe) is capable of hybridizing to complementary DNA (C-uidA) from the bacteria of interest. Non-complementary DNA sequences (NC-uidA) will not hybridize to the probe on the electrode surface. The oligonucleotide probe can be designed to be specific for other genes of E. coli or other microorganisms of interest.

Hybridization of the uidA gene oligonucleotide targets onto the Pt-PPY-oligo electrode. The electropolymerization was achieved by a continuous cyclic voltammetry scanning for twenty-six (26) cycles between 0.0 and +0.70V at a scan rate of 50 mV/s. Electro-polymerization of 0.05M PPY was achieved using 0.5 M KCl as electrolyte following previous protocols (Wang, J., et al., Analytical Acta 402: 7-12 (1999)). Background signal was performed by cyclic voltammmetry with a blank electrolyte solution of 0.25M NaCl.

Sensitivity analysis of the E. coli DNA biosensor: Hybridization with complementary twenty-five base pair (25 bp) oligonucleotide target. Hybridization experiments were carried out with a 25 bp complementary (SEQ ID NO:6) and 25 bp non-complementary (SEQ ID NO:7) oligonucleotides to the uidA gene. The hybridization solution consists of 2 ml 0.25M NaCl and was also used as a blank solution for measurement of background signal. A working potential of +0.7V was applied for fifteen (15) seconds and allowed to decay for sixty (60) seconds previous to the spiking of the non-complementary sequence. The electrochemical response was measured using the Versastat II potentiostat/galvanostat to generate cyclic voltammograms. Voltammograms of current (I) vs. potential (V/Ag/AgCl) were used to evaluate the performance of the biosensor under the standard DNA concentration. Functionality analysis were performed using a standard concentration for target and non-target DNA (25 bp oligonucleotides) of 10⁻⁶ grams (10 μg) of total DNA. Hybridization experiments were held using different times of hybridization (30, 60 and 180 minutes). Distinctive subtractive hybridization signals were obtained for the complementary oligonucleotides after 180 minutes of incubation period at room temperature. Subtractive voltammograms were generated taking into consideration the background signal from the signal during electro-deposition and hybridization events.

Results and Discussion: Incorporation of a uidA gene oligonucleotide into a conductive polymer-electrode biosensor system: a polypyrrole-coated platinum electrode.

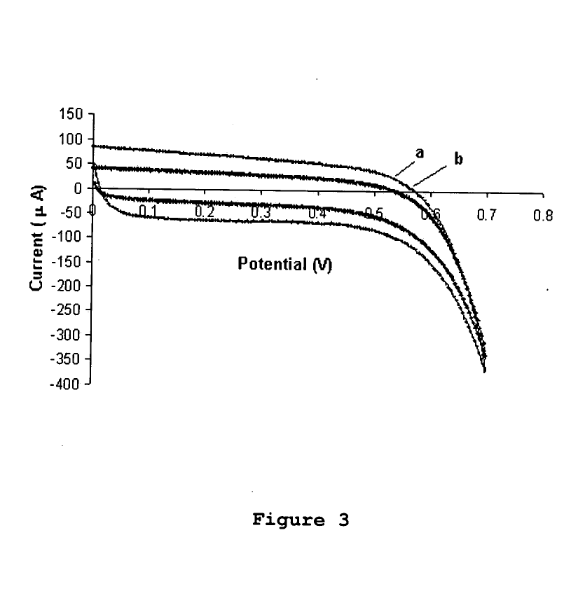

Electropolymerization of PPY: Successful electropolymerization of the conductive polymer was achieved using a 0.05M PPY/0.5 M KCl solution. FIG. 3 shows characteristic cyclic voltammograms of the electropolymerization of PPY onto Pt. The current for cycle 26 (a) was higher than that for cycle 13 (b) indicating a successful deposition on the Pt. All CVs were recorded using a potential between 0.0 and 0.7 V at a scanning rate of fifty millivolts per second (50 mV/s).

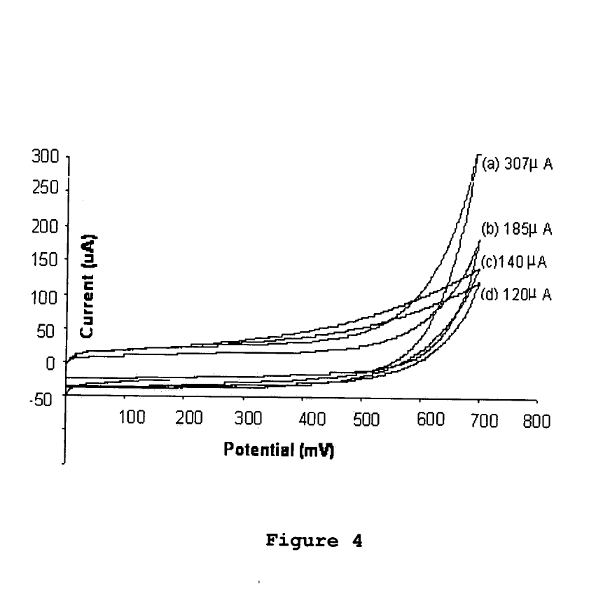

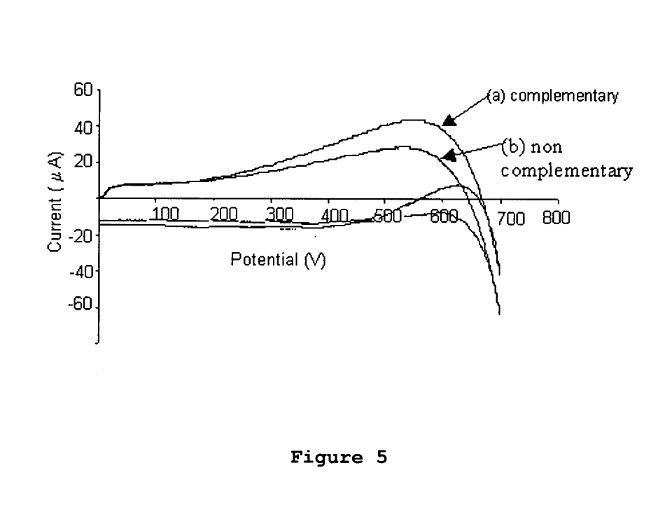

Functionalization of the Pt-PPY-uidA biosensor: The preparation of the modified Pt-PPY-uidA electrode biosensor was achieved by electrodeposition of 0.05M PPY with 1 μg of a twenty-five base pair (25 bp) uidA oligonucleotide probe. The same amount of total DNA was used for both the complementary oligonucleotide specific for E. coli uidA gene and the non-complementary oligonucleotide. After PPY-DNA electrodeposition, the application of potential was suspended for 15 minutes. During that time the spiking of non-complementary was followed by complementary target oligonucleotides. The cyclic voltammograms for a blank solution, electrodeposition process and hybridization with complementary and non-complementary oligonucleotides are demonstrated in FIG. 4. The electrolyte solution over bare platinum shows a current peak of 307 μA and the current peak for background during the electrodeposition event is 185 μA. There is a clear change in the current after hybridization of the complementary oligonucleotide (140 μA) and for non-complementary oligo (120 μA). The drop in current after each event is clearly distinguishable from one another. Subtractive cyclic voltammograms of hybridization signals from the background (FIG. 5) show a clear difference in the hybridization process between complementary and non-complementary sequences of DNA using the standard concentration of 1 μg/μl of synthetic probes. There is a clear difference in the current peaks for complementary sequence at 46 μA and for the non-complementary sequence at 27 μA at a potential of 567 mV. These results show a clear distinction in hybridization versus non-hybridization signal with this DNA concentration. The formation of a hybrid due to the recognition of the probe to the complementary sequence means a successful transfer of electrons along the dsDNA chain to the conductive PPY. This explains the higher current output signal than that obtained for the non-complementary reaction.

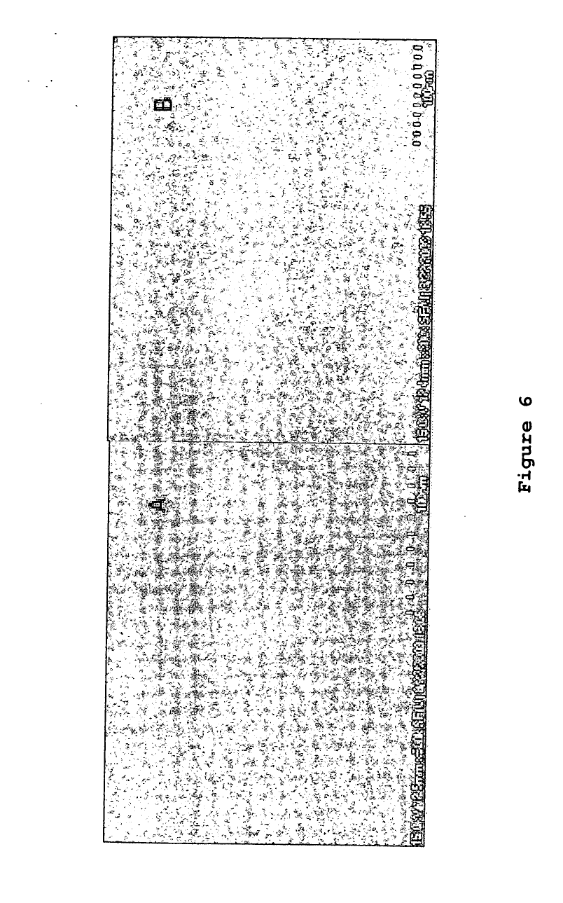

Physical Characterization of the modified DNA-PPY electrode surface: Scanning Electron Microscopy. The Scanning electron microscopy (SEM) images of the bare (A) and modified (B) Pt electrodes are shown in FIG. 6. The bare Pt surface (A) has a smoother surface than the PPY coated Pt surface (B) indicating adequate modification of the working electrode. FIG. 6B shows the modified PPY-PT-DNA surface. The dark regions on the modified surface appear to be the positively charged polarons where the DNA probes are doped within the PPY. Hydrogen bonds between the PPY and the oxygen molecules from the phosphate group in the backbone of the DNA chain allow this embedding. The resolution of the SEM image (100 nm) is not high enough to clearly show the DNA structure.

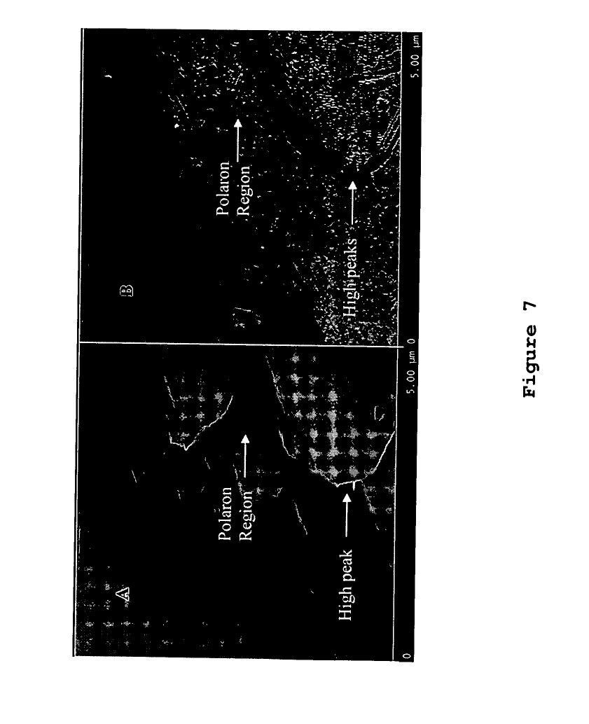

Atomic Force Microscopy. Atomic Force Microscopy (AFM) was taken to demonstrate the polymerization of 0.05M pyrrole into the Pt electrode. AFM experiments were performed with a NanoScope IIIa from Digital Instruments (Santa Barbara, Calif.) with the tapping mode. FIG. 7 shows a two-dimensional surface profile of the PPY after applying 700 mV at a scan rate of 50 mV/s. The surface profile images were taken prior to the functionalization of the PPY, which was embedded with a 25 bp oligonucleotide. A 5 μm section of the film can be observed. Both images show the same area of the Pt electrodes at different contrast. Side A of the pictures shows the gaps between polymer structures called polarons.

The polaron dimensions varied in magnitude from 0.5 μm to approximately 1.0 μm in size. The demonstration of the polaron areas is of great significance since that is the region where the 25 bp oligonucleotide becomes embedded after the functionalization of the PPY film (Pande 1998). The same area of the polaron can be appreciated in part B of the image using a different contrast. A variation in surface roughness was observed and different peak heights were detected. The highest peaks observed had a height of 2000 nm. The AFM images have demonstrated the distribution of positive polaron regions after electropolymerization of PPY. This phenomenon facilitated the immobilization of the anions in the form of the 25 bp uidA probe. It has been demonstrated that negative charges of the DNA probe are required to counteract with the positive charges of the polymer backbone and alter charge neutrality (Shidmidzu, 1987).

Specificity of the uidA probe: The specificity of the biorecognition was demonstrated with the use of a synthetic 25 bp oligonucleotide specific for E. coli uidA gene sequence. Specificity was demonstrated after distinctive signals of complementary sequence yielded a higher current signal than that obtained for a non-complementary sequence.

The synthesis of the functionalized electrode and hybridization events took place for a total period of three hours. However, the functionalization of the electrode, which is the bulk of the work, can be done offline. Hybridization can be performed in less than thirty (30) minutes. For example, when manufactured in high quantities, the functionalized electrodes can be stored and used for hybridization purposes in a time frame of fifteen to twenty (15-20) minutes. This is a significant reduction in time from regular detection or culture techniques (24 to 48 hrs). This represents an incredible reduction of detection time, which will be ideal under a bioterror attack situation or in the event where rapid water quality monitoring is needed.

A single-stranded oligonucleotide DNA (ssDNA) biosensor was successfully designed and fabricated. The modified working electrode Pt-PPY-uidA probe was functionalized using electrodepositon techniques. A DNA concentration of 1 μg was sufficient to detect hybridization events. The hybridization event was clearly distinguishable from non-complementary sequence using cyclic voltammetry techniques. The hybridization event was detected after a short period of incubation (15 min). This demonstrates the great potential of the DNA based biosensor as a viable tool for rapid biosensor response and its possible use in water quality and other events where rapid detection might be needed.

Functionality, selectivity and sensitivity of the DNA biosensor using different concentrations of oligonucleotides.

Optimization of E. coli biosensor using different ionic strength electrolytes: Polymerization experiments were carried out using different concentrations of NaCl as electrolyte to produce a distinctive cyclic voltammogram. We were able to determine the optimum ionic strength of the electrolytic solution. After several cyclic voltammograms using 0.1M, 0.2M and 0.25 M NaCl, the 0.25M concentration was the most effective in the generation of cyclic voltammograms that reflected the polymerization of pyrrole and the incorporation of the uidA gene into the polymer film. Lower concentrations of NaCl failed to produce a clear CV. An optimum electrolytic concentration that could both enable the production of good quality PPY films and not affect the hybridization time was essential in this study. The use of low ionic strength electrolyte solutions has proven not to be the best option to produce high quality PPY films (Wang 1999). The ionic strength of 0.25M NaCl has been reported in literature as an adequate ionic strength where the event of hybridization is still in effect at low temperatures close to 25° C. (Piunno, Watterson et al. 1999). It has been demonstrated that DNA adsorption into PPY increases with increasing ionic strength. The optimum adsorption range was found to be from 0.1M to 0.3M (Saoudi, Jammul et al. 1997). The effect of several ionic strength solutions in the immobilization of DNA oligonucleotides in sensor surfaces was also demonstrated (Watterson, Piunno et al. 2002). A low ionic strength of 0.25M corresponded to a high immobilization density of probes into the biosensor. This could be due to a decrease in electrostatic repulsion between DNA molecules.

After determination of the optimum ionic strength for the generation of CV profiles, we proceeded to determine the effect of the hybridization temperature in the generation of cyclic voltammetry signals. The melting temperature of the probe (Tm) used was calculated to be 72° C. No difference in CV profiles was observed at this ionic strength from 72° C., 64° C. and room temperature (RT). We decided to continue all incubation periods at room temperature.

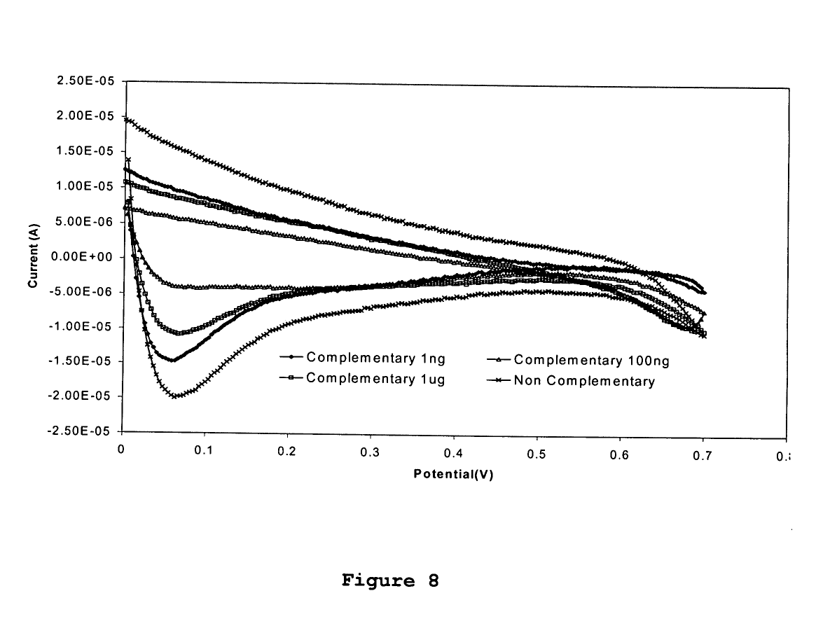

Concentration effects were also taken into consideration for the functionalization of the biosensor. After incubation of the 10⁻⁶ g to 10⁻⁹ g oligonucleotide probe/0.25M NaCl with the functionalized PPY film, we obtained the cyclic voltammograms that can be seen in FIG. 8. In this figure we can compare the CV for different concentrations of the total oligonucleotides ranging from 10⁻⁶ to 10⁻⁹ g after subtraction of the background signal. Background signals (not shown) were registered in the same current range than those for non-complementary signals. These results demonstrated that non-complementary targets did not bind to the uidA probe embedded in the PPY film. All CVs displayed a decreased current at potentials beyond 0.6V. The drop in the current beyond this potential corresponded to the over oxidation of the PPY film. Previous evidence have demonstrated that at these positive potential ranges, there is a loss of the π-electron network and film conductivity (Mostany and Scharifker 1997) causing the over oxidation peaks. The DNA oligonucleotide did not undergo any oxidation at the potential range used. The peak that can be observed around the 0.1V potential may have been an effect of the background subtraction and did not correspond to any redox activity by the DNA. We can then appreciate a small difference with the 1 ng concentration, followed by the 1 μg concentration. The biggest difference in the current after subtraction from background was obtained with the 100 ng concentration. After these results we concluded that the optimum concentration to determine the biggest difference in current from background signal corresponded to the 100 ng total concentration. The current range seemed to decrease with the decrease in oligonucleotides concentration with the exception of the 1 ng concentration. The 1 ng concentration CV profile laid very close to the background signal, suggesting that this concentration was too low for an accurate identification of the hybridization event. The macro scale of the biosensor could have caused this concentration limitation. This detection limit is probably not as low as desired for a commercial biosensor. The detection limit situation could be solved by a reduction of the Pt-PPY-uidA biosensor scale. The use of microelectrodes and microelectronic devices could be a potential future scope in solving the detection limit factor. The use of nano scale electrochemistry could also be of advantage to this detection limit factor. Lower detection limits in the range of femptomoles have been obtained in other DNA based biosensor at a much lower scale and with the use of nanoparticles (Wang 2003). The CV signals using 1 μg of complementary sequences were estimated to be distinctively different from the background and the non-complementary ones as well as different from the CV using 100 ng of sample. The decrease in current after hybridization with complementary oligonucleotides have also been reported previously (Korri-Youssoufi 1997). This phenomenon may be the result of the increased charge density generated by the formation of the double strand DNA.

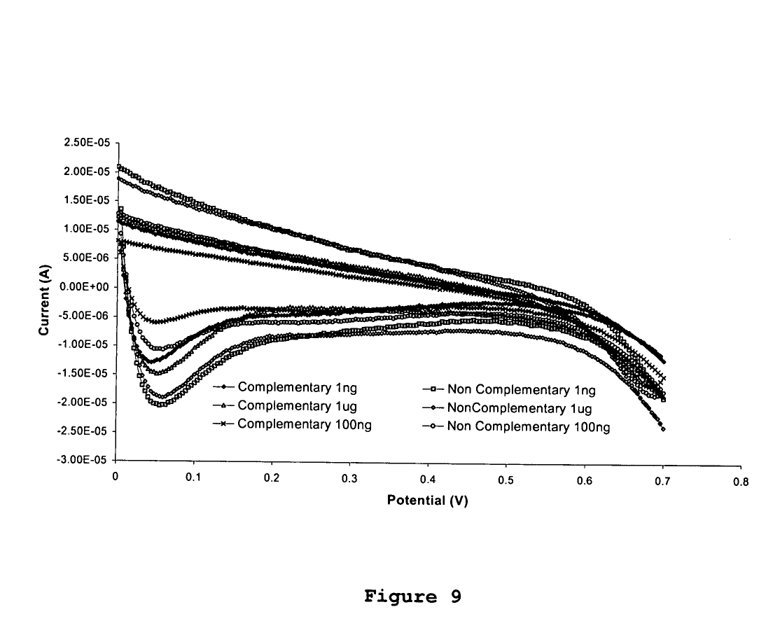

FIG. 9 shows the difference of complementary signals from its correspondent non-complementary ones. All of these signals are distinctively different from background signals. Background signals were comparable in dimensions to those obtained for non-complementary targets. Background signals have not been shown for simplicity purposes. The analysis also demonstrated a great variability within background signals. Therefore by subtraction of background signals from actual signals, variability was introduced to the resulting CVs. We proceeded to analyze the actual CV signal without the background to reduce the variability factor introduced by them.

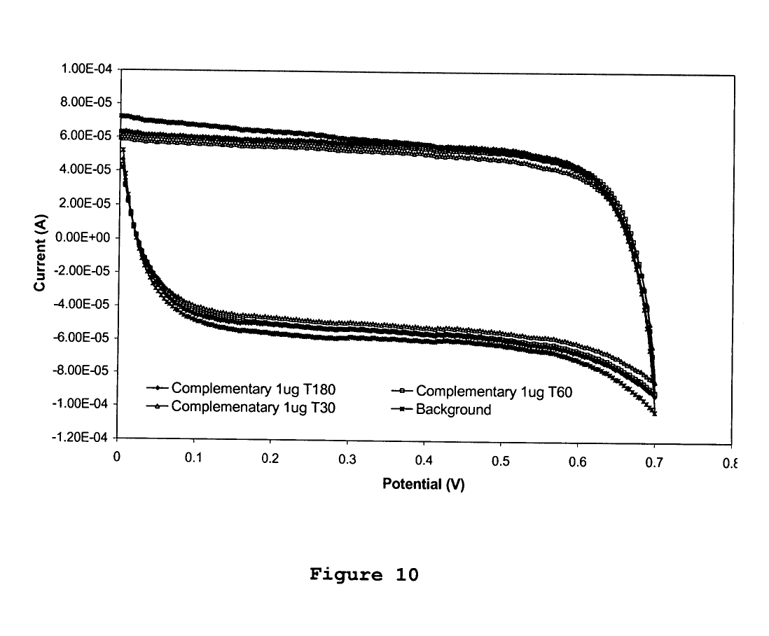

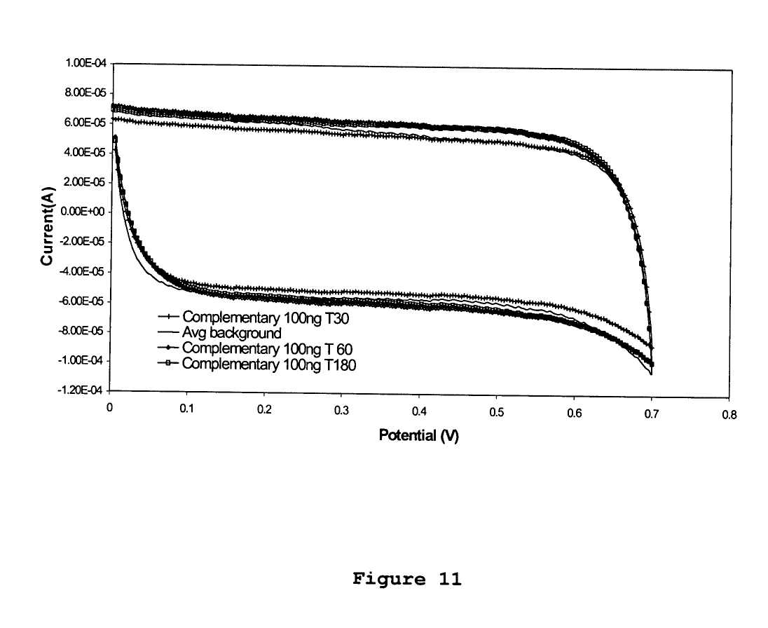

The results of the CV signals without subtraction from background signals are shown in FIG. 10 for 1 μg of complementary and non-complementary probes. At this particular concentration of oligonucleotides, there was only a significant difference from the background signal and no difference was observed for different hybridization times. FIG. 11 shows the effect of hybridization time at a 100 ng concentration of complementary and non-complementary probes against the background signal. A significant difference can only be appreciated after 30 minutes of hybridization time. These results are confirmed by statistical analysis at 95% confidence that can be compared in Table 4 and will be discussed in the following section.

The higher current range demonstrated by background and non-complementary probe solutions might correspond to the doping of both the Cl— anion and the negatively charged DNA into available polaron sites that are not occupied by the 25 bp probe. This induces a flow of electron transfer along the PPY film, resulting in CV profiles with higher current output. Hybridization events and the formation of a double strand (ds) after hybridization might cause an obstruction of the π-electrons from the dsDNA to the PPY resulting in CVs with a reduced current range output. According to DNA adsorption kinetics studies, 85% of DNA used was adsorbed into PPY after 10 minutes and total equilibrium of adsorption kinetics were achieved in less than 45 minutes (Saoudi, Despas et al. 2000). These results, along with the results from our statistical analysis, supported our decision to use 30 minutes for hybridization incubation times.

Delta Charge (ΔC) analysis for the normalization of CV signals: Electrochemical analyses using cyclic voltammetry for DNA hybridization studies do not exhibit the typical CV graphs with evident cathodic and anodic peaks from reversible redox reactions. DNA does not undergo a redox reaction at the potential range used for these studies. Therefore, a more in depth analysis of the CV was obtained using delta charge value (ΔQ), which represents the integral of current across the selected set of points with respect to time. The ΔQ value was expressed in milliCoulombs (mC) and was chosen to normalize the area under the curve that represents the totality of the 383 data points obtained in every CV. It is also an analytical tool that permits comparison of the change in the current as a result of the hybridization process.

Besides the subtractive CVs, analysis of variance (ANOVA) of the delta charge value was obtained to determine the statistical significance of the different experimental conditions. Table 2 summarizes the parameters used for the statistical analysis with 95% confidence using ANOVA. The parameters tested were, the melting temperatures (Tm) (72° C., 64° C. and RT), two technical replicates (cycles) and three biological repetitions. For the 1 μg concentration we were able to obtain a significant difference only for the background signal against its corresponding signal. All CV signals were demonstrated to be significantly different from their correspondent background using a type 3 test of fixed effects in the ANOVA analysis (P=0.042). These results were summarized in Table 3. The different hybridization temperatures did not affect the hybridization event using 1 μg of total oligonucleotides.

Table 4 presents the average value of ΔQ in mC for different concentrations of probes (1 μg and 100 ng) at different hybridization times (30, 60, and 180 minutes). The percentage of change for the ΔQ value varied from one concentration to the other as well as hybridization times. It was observed that for both probe concentrations the highest change in charge was for the complementary sequence after 30 minutes incubation time.

TABLE 2
Parameter levels for ANOVA Mixed
Procedure Analysis.

| Class | Levels | Parameters |
|---|---|---|
| Signal | 3 | Background |
| | | Complementary |
| | | Non- |
| | | Complementary |
| Concentration | 1 | 1 μg |
| Conditions | 3 | 64° C., 72° C., RT |
| Replications | 3 | 1, 2, 3 |
| Cycles | 2 | 13, 26 |

TABLE 3
Type 3 Tests of Fixed Effects

| | Num | | F | |
|---|---|---|---|---|
| Effect | DF* | Den DF | Value | Pr > F |
| Signal | 2 | 26 | 11.26 | 0.0003 |
| Background | 1 | 4 | 0.73 | 0.4416 |
| Signal *Backg | 2 | 26 | 6.80 | 0.042 |

*DF = degrees of differences.
Conditions used was actual signal vs. background

TABLE 4
Average ΔQ at different times and
Concentrations for uidA probes

| | Hybridization | | | |
|---|---|---|---|---|
| | Time | Average | Average ΔQ | % of Q |
| Signal Type | (minutes) | ΔQ (mC) | (mC) | change |
| | | 1 μg | 100 ng | |
| Complementary | T180 | −40.46 | −24.88 | 38% |
| | | ±5.52 | ±7.12 | |
| | T30 | −54.65 | −63.62 | 14% |
| | | ±5.52 | ±7.12 | |
| | T60 | −43.31 | −26.59 | 38% |
| | | ±5.52 | ±7.12 | |
| Non | T180 | −50.91 | −41.46 | 18% |
| Complementary | | ±5.52 | ±7.12 | |
| | T30 | −50.70 | −117.33 | 57% |
| | | ±5.52 | ±7.12 | |
| | T60 | −42.78 | −37.84 | 12% |
| | | ±5.52 | ±7.12 | |

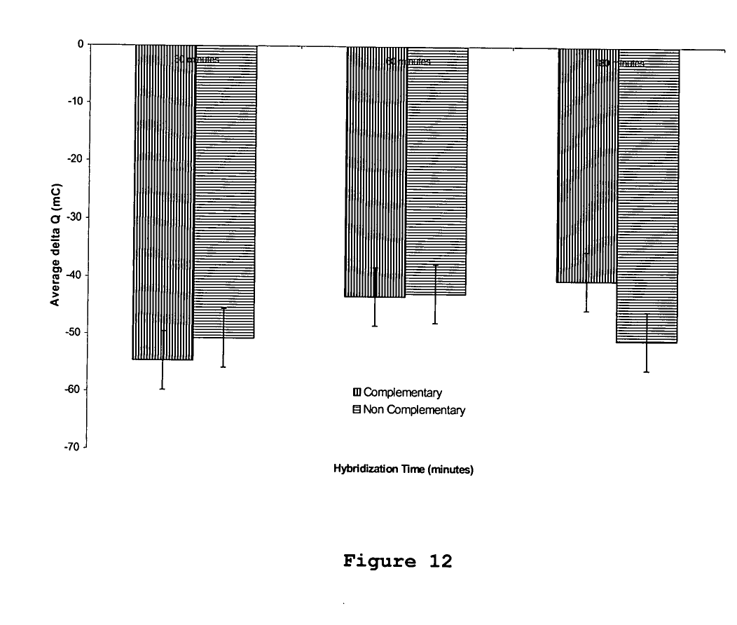

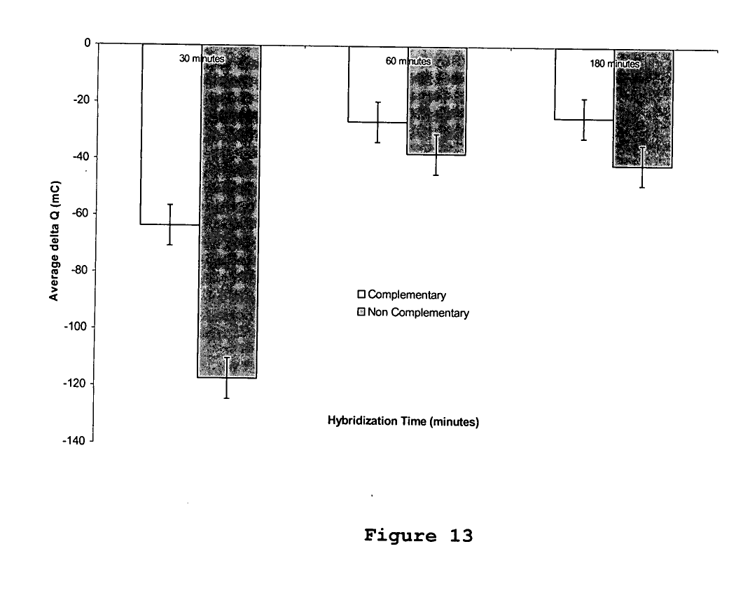

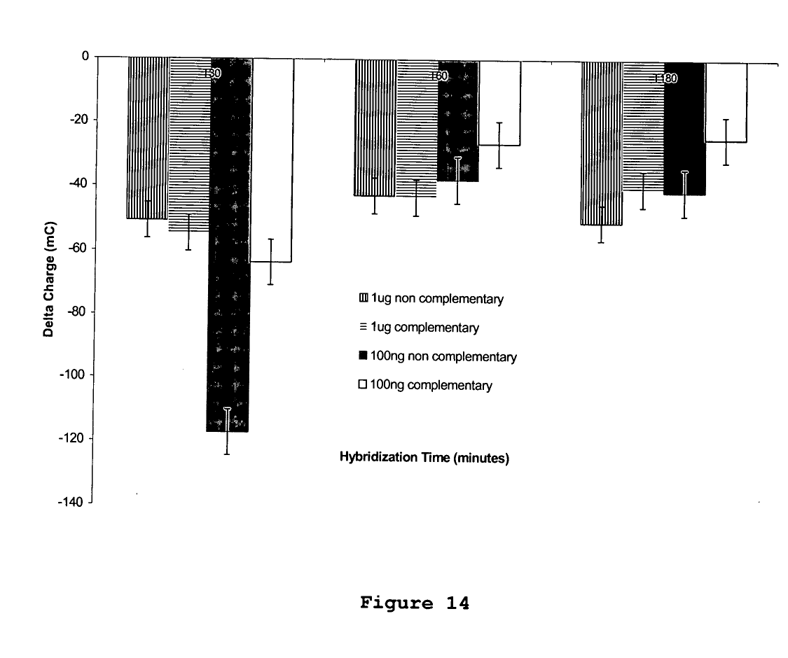

The highest ΔQ value obtained was the one for the non-complementary signal: (−117.33±7.12 mC) after 30 minutes of hybridization period. This value was especially important since it yielded a close value to background signal (−118.22±13.23 mC). The high values for both the background and the non-complementary signal corresponded to the doping of the PPY polaron regions that were not occupied by the 25 bp probe. The doping of the Cl— anion interacted with the conductivity of the PPY making it more electroactive. These results can be better observed in FIG. 12 where average ΔQ values are compared for 1 μg of complementary and non-complementary oligonucleotides after 30, 60 and 180 minutes of hybridization time. There was not a statistically significant difference in ΔQ values at any of the hybridization times for this particular concentration. The same studies were performed using a concentration of 100 ng of complementary and non-complementary oligonucleotides during 30, 60 and 180 minutes of hybridization time. FIG. 13 shows the ΔQ mean values for 100 ng of complementary and non-complementary probes. In this case, ANOVA analysis confirmed the statistically significant difference between hybridization times at this particular concentration after 30 minutes of hybridization time. After 30 minutes of hybridization, the change in charge value was −63.62±7.12 mC for the complementary target. This represented a 60% decrease in ΔQ value after 60 and 180 minutes of hybridization time. Comparative ΔQ values from both concentration and hybridization times are summarized in FIG. 14. The hybridization of different concentrations of the complementary oligonucleotide affected the electroactivity of the PPY film and a change in charge was observed in the range of −63.62±7.12 mC for 100 ng of complementary probe vs. −54.65±5.52 mC for 1 μg complementary probes after 30 minutes of hybridization time. This represented only a 14% increase in the complementary oligonucleotide hybridization signals from 1 μg to 100 ng. In contrast, the value of ΔQ decreased 38% for lower concentrations after longer periods of hybridization times (60 and 180 minutes). Note the high value (−117.33±7.12 mC) for non-complementary probe signal after 30 minutes of hybridization. This value was very similar from the background value of −118 mC. This corresponded to a 46% change in ΔQ value for the 100 ng complementary probe after 30 minutes of hybridization time. The background ΔQ value was not included in the graph for simplification reasons.

The most statistically significant different value corresponded to the one obtained after 30 minutes of hybridization time. The negative value of the integral corresponded to the net negative charge of the DNA probes and therefore was observed at the anodic portion of the CV.

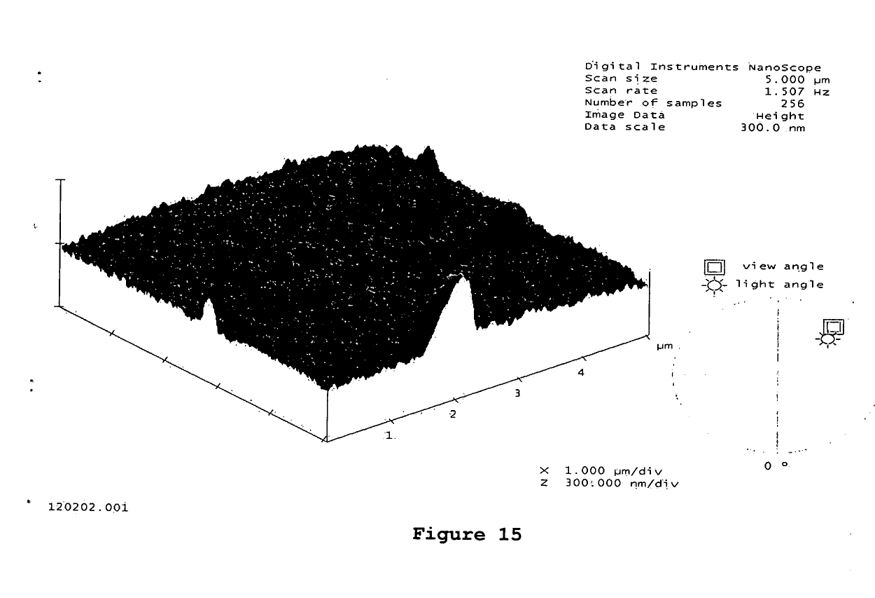

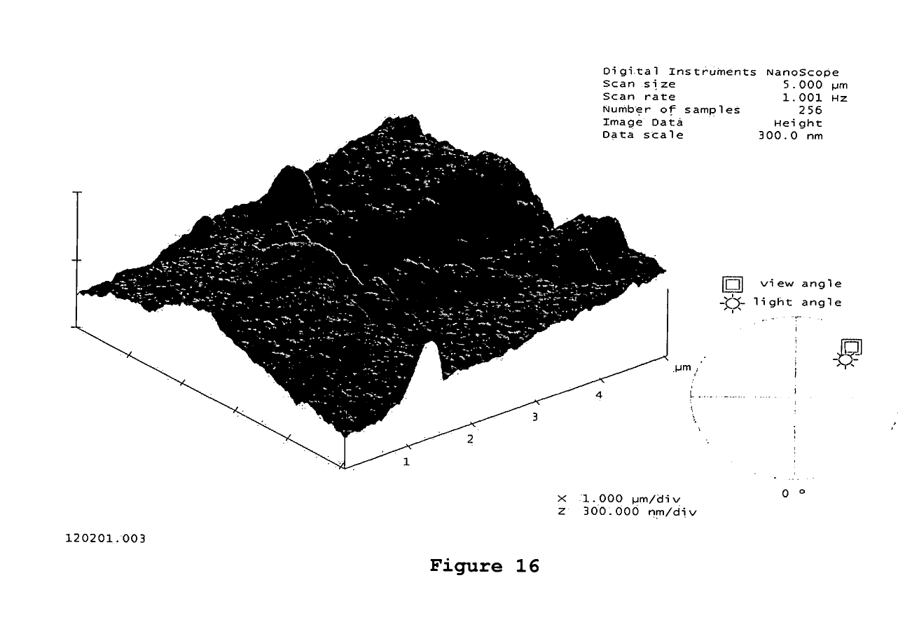

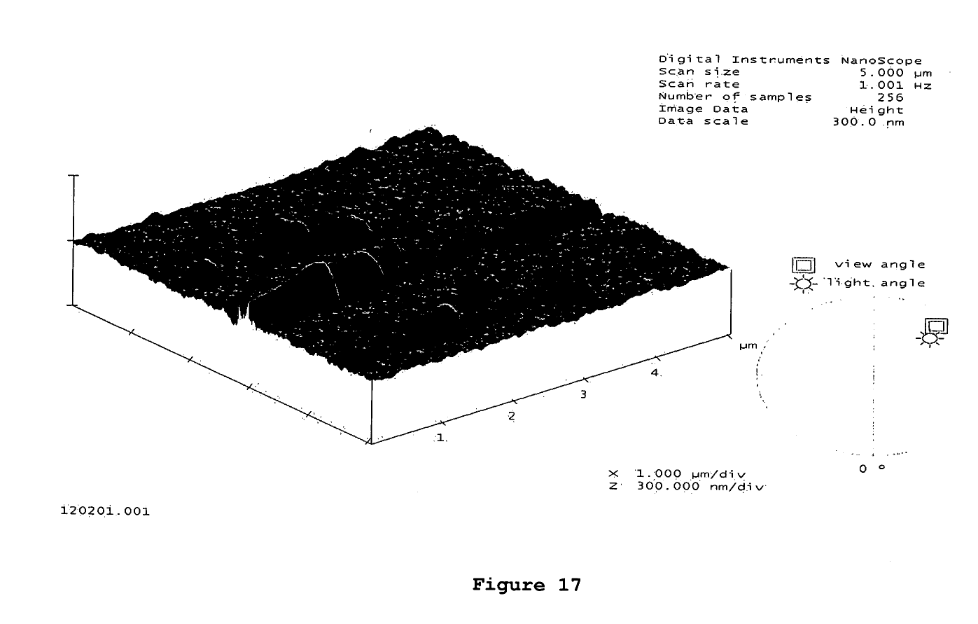

AFM Characterization of the Pt-PPY-uidA Biosensor. The Pt-PPY biosensor was embedded and functionalized with the 25 bp uidA probe specific for E. coli directly into the polaron regions of the PPY film. Confirmation of the embedding process was obtained using atomic force microscopy (AFM). FIGS. 15-17 show the surface profiles of the Pt-PPY film prior to DNA immobilization, after functionalization and after hybridization with complementary uidA target oligonucleotide respectively. FIG. 15 shows a surface profile relatively smooth with occasional rough areas. This variation in roughness, peaks and valleys, is a common property of PPY films (Pande 1998). FIG. 16 shows the AFM image of the functionalized biosensor where a more rough surface profile has been created due to the embedding of 1 μg of the uidA probe into the PPY film. The increase in the surface roughness is a positive aspect because it creates more surface area for the hybridization process to occur. This variation in surface roughness could have been one of the causes for the change in conductivity properties of the PPY film. The variation in the surface profiles might facilitate the DNA embedding into the film. FIG. 17 shows the change of the surface profile after 30 minutes of hybridization with 100 ng of complementary uidA target oligonucleotide. Smoother regions were observed after the formation of double strand (ds) DNA by the hybridization process. The presence of large polyelectrolyte dopants (in this case, ds DNA) induced a reduction on the conductivity of PPY films. This phenomenon has been reported by several researchers (Davey, Ralph et al. 1999). Large anions can be of influence on the chain packing of the PPY film by increasing inter-chain hopping distances. This could be a possible explanation of the lower CV profiles after hybridization with complementary oligonucleotides in our study.

We have successfully determined the optimum conditions for the performance of the Pt-PPY-uidA biosensor using 25 bp complementary and non-complementary oligonucleotides. We determined the use of 0.25M NaCl as an optimum hybridization solution for the creation of distinctive CVs. We were able to observe that the use of a total of 100 ng of uidA complementary oligonucleotides was the optimum concentration for the creation of statistically significant different CVs. We also determined the optimum hybridization time to be 30 minutes at room temperature to obtained successful and distinctive CVs. The successful creation and functionalization of the Pt-PPY-uidA was confirmed using AFM.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A biosensor electrode for the detection of the presence in a water sample from a drinking water or food source of a nucleic acid from a bacteria of interest comprising:
(a) an electrode;
(b) polypyrrole electropolymerized on the electrode; and
(c) an oligonucleotide as a dopant bonded to the polypyrrole having an oligonucleotide nucleotide sequence, wherein at least a portion of the oligonucleotide sequence is complementary to a unique target sequence of the nucleic acid from the bacteria of interest such that the nucleic acid from the bacteria hybridizes to the oligonucleotide for detecting the presence of the bacteria of interest by a change in conductivity of the electrode.

2. The biosensor electrode of claim 1 wherein the target nucleic acid is DNA or RNA.

3. The biosensor electrode of claim 1 wherein the oligonucleotide comprises a DNA sequence from the uidA gene of E. coli.

4. The biosensor electrode of claim 1 wherein the electrode is platinum.

5. A biosensor system for the rapid detection of the presence in a water sample from a drinking water or food source of a nucleic acid from a bacteria of interest comprising:
(a) an electrode;
(b) polypyrrole electropolymerized on the electrode;
(c) an oligonucleotide as a dopant bonded to the polypyrrole having an oligonucleotide nucleotide sequence, wherein at least a portion of the oligonucleotide sequence is complementary to a unique target sequence of the nucleic acid from the bacteria of interest such that the nucleic acid from the bacteria hybridizes to the oligonucleotide for detecting the presence of the bacteria of interest by a change in conductivity of the electrode; and
(d) an electrical detection apparatus, wherein when the oligonucleotide hybridizes to the nucleic acid an electrical signal as a result of a change of conductivity of the electrode is generated which is used to detect the presence of the bacteria of interest in the water sample.

6. The biosensor system of claim 5 wherein the target nucleic acid is DNA or RNA.

7. The biosensor system of claim 5 wherein the oligonucleotide comprises a DNA sequence from the uidA gene of E. coli.

8. The biosensor system of claim 5 wherein the electrode is platinum.

9. The biosensor system of claim 5 wherein the detection apparatus is a potentiostat.

10. A method of using a biosensor system for rapidly detecting the presence in a water sample from a drinking water or food source of a nucleic acid from a bacteria of interest comprising:
(a) providing a biosensor system comprising an electrode, a polypyrrole electropolymerized on the electrode, an oligonucleotide as a dopant bonded to the polypyrrole having an oligonucleotide nucleotide sequence, wherein at least a portion of the oligonucleotide sequence is complementary to a unique target nucleic acid sequence of the nucleic acid from the bacteria of interest such that the nucleic acid hybridizes to the oligonucleotide when detecting the presence of the bacteria of interest, and a detection apparatus;
(b) providing a water sample to be tested;
(c) adding a lysis solution to the water sample so as to rupture any bacteria present in the water sample to provide a prepared water sample;
(d) providing the prepared water sample to the electrode of the biosensor system;
(e) generating a signal with the detection apparatus; and
(f) analyzing the delta charge value (ΔQ) of the signal so as to detect whether the nucleic acid from the bacteria of interest is present in the water sample.

11. The method of claim 10 wherein the target nucleic acid is DNA or RNA.

12. The method of claim 10 wherein the oligonucleotide comprises a DNA sequence from the uidA gene of E. coli.

13. The method of claim 10 wherein the electrode is platinum.

14. The method of claim 10 wherein the detection apparatus is a potentiostat generating a cyclic voltammogram.
